(19) United States
(12) Patent Application Publication
(10) Pub. No.: US 2026/0022092 A1
(43) Pub. Date: Jan. 22, 2026

(54) PROCESS FOR SOLVENT FREE CONTINUOUS SYNTHESIS OF AMIDES AND PEPTIDES

(71) Applicant: Council of Scientific and Industrial Research, New Delhi (IN)

(72) Inventors: Amol Arvindrao KULKARNI, Pune (IN); Ranjit Shabu ATAPALKAR, Pune (IN)

(21) Appl. No.: 18/992,687

(22) PCT Filed: Jul. 12, 2023
(86) PCT No.: PCT/IN2023/050681

§ 371 Date: Jan. 9, 2025

(30) Foreign Application Priority Data
Jul. 12, 2022 (IN) 202211040157

(51) Int. Cl. C07C 231/02; B01J 19/20; C07K 1/10

(52) U.S. Cl. CPC C07C 231/02; B01J 19/20; C07K 1/10

(57) ABSTRACT

The present invention relates to a novel process for the solvent free continuous synthesis of amides and peptides. More particularly, the invention relates to a process for synthesizing amides and peptides using coupling reagents where the process is done at room temperature with lower residence time and solvent free synthesis, producing enhanced yields and purity products.

### FIELD OF THE INVENTION

The invention relates to a novel process for the solvent free continuous synthesis of amides and peptides. Particularly, the invention relates to a process for synthesizing amides and peptides using coupling agents where the process is done at room temperature with lower residence time and solvent free synthesis, producing enhanced yields and purity products.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The amide and peptide are universal functional groups in nature. Amide and peptides are building blocks of natural products, peptides, and protein synthesis. Amide and peptide bond formation serves as a fundamental reaction in chemistry, and is practically useful for the synthesis of macromolecules, complex peptides, food additives, and polymers and many more. Amide is not only a fundamental structural unit of peptide and protein but is also widely found in pharmaceuticals, agrochemicals, pesticides, polymers, fine chemicals, and other materials. A straightforward synthetic strategy for amide bond formation is the coupling of carboxylic acid and amine using coupling reagents. In the conventional batch protocol, the majority of amide bond syntheses involve the use of stoichiometric amounts of a metal catalyst, bulk solvents, and long reaction time, and energy consuming, making them generally expensive and wasteful procedures.

The amides and peptides are synthesized employing harsh metal catalysts, long reaction times, high energy requirement and huge amount of solvents to yield 60-95%. However, for pharmaceutical and fungicides applications as in the case of amide and peptides, highly pure product is desired. Conventionally this reaction of acids and amines treated with coupling reagents by batch method at high temperature. But in large scale operations involving very large quantities of solvents, significant limitations on recovery of solvents and further distillation are encountered. In large scale operations, high temperature processes are also not very desirable as it can affect purity while reusing them for such products.

So, there is a need in the art to provide a process of solvent free continuous synthesis of amides and peptides to be conducted at low temperatures, preferably at room temperature. It would be an added industrially advantageous feature of the process when the same provides a pure product in high yields without using solvent.

Thus, this invention meets the need for the improved methods of amides and peptides synthesis for arriving at a sustainable approach, including those related to solvent free continuous synthesis, allowing production of higher amounts of amides and peptides than the known solvent free methods.

### OBJECTS OF THE INVENTION

An objective of the invention is to provide a solvent free continuous process for synthesis of amides and peptides at room temperature.

Another objective of the invention is to provide a solvent free room temperature process for the synthesis of amides and peptides with high purity such that there is no requirement of further purification processes.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

The present disclosure discloses a solvent free continuous process for the synthesis of peptides and amides includes reacting an acid with an amine in the presence of a coupling agent in a single screw reactor at a temperature in the range of 20-35° C. for a residence time in the range of 10 seconds to 300 seconds to obtain peptides and amides.

The present invention provides a process of solvent free continuous synthesis of amides and peptides in a screw reactor with greater than 95% conversion of the solid/liquid substrates and 49-95% yield of the desired products.

Accordingly, in an aspect, the present invention relates to a continuous process for the synthesis of peptides and/or amides comprising:
reacting an acid with an amine in the presence of a coupling agent in a single screw reactor at a temperature in the range of 20-35° C. for a residence time in the range of 10 seconds to 300 seconds to obtain the peptides and/or amides.

In an embodiment, the acid used in said process is selected from aliphatic acid and/or aromatic acid.

In another embodiment, the acid is a mono acid, a di-acid or a poly acid.

In another embodiment, the amine is selected from primary amine and/or secondary amine.

In another embodiment, the coupling agent is selected from a group consisting of 1-(3-dimethylaminopropyl)-3-ethylcarbodiimide hydrochloride (EDC.HCl), N, N′-dicyclohexyl carbodiimide (DCC), 1-hydroxybenzotriazole (HOBt), 1,1′-carbonyldiimidazole (CDI), N, N′-diisopropylcarbodiimide (DIC) and combination thereof.

In another embodiment, the single screw reactor rotates at a speed in the range of 20-250 rpm.

In another embodiment, the conversion rate of the solid/liquid substrates into the amides and/or peptides is in range of 95 to 100%. The solid/liquid substrate is amine and/or acid.

In another embodiment, the yield of the peptides and/or amides is in range of 45 to 95%.

In another embodiment, the yield of the peptides and/or amides is in range of 49 to 95%.

In another embodiment, the purity of the amides and/or peptides is in range of 95 to 99%.

In another embodiment, the continuous process as claimed is a solvent free continuous process.

In yet another embodiment, the continuous process as claimed is a purification free continuous process.

### BRIEF DESCRIPTION OF THE DRAWINGS:

FIG. 1 shows Proton NMR of (3-methyl-N-phenylbenzamide) (compound 1y).

FIG. 2 shows ¹³C NMR of (3-methyl-N-phenylbenzamide) (compound 1y).

FIG. 3 shows Proton NMR of compound 1w [methyl (tert-butoxycarbonyl)-L-valyl-L-phenylalaninate)].

FIG. 4 show ¹³C NMR of compound 1w [methyl (tert-butoxycarbonyl)-L-valyl-L-phenylalaninate)].

FIG. 5 shows the representative drawing of the single screw reactor as used in the present invention.

FIG. 6 shows the general scheme for the synthesis of amides and peptides.

### ABBREVIATIONS USED

EDC.HCl: 1-(3-Dimethylaminopropyl)-3-ethylcarbodiimide hydrochloride
DCC: N, N′-Dicyclohexylcarbodiimide
HoBt: 1-Hydroxybenzotriazole
CDI: Carbonyldiimidazole
DIC: Diisopropylcarbodiimide

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it is individually recited herein.

All processes described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

Various terms as used herein are shown below. To the extent a term used in a claim is not defined below, it should be given the broadest definition persons in the pertinent art have given that term as reflected in printed publications and issued patents at the time of filing.

An embodiment of the present disclosure discloses solvent free continuous process for the synthesis of peptides and amides comprising: reacting an acid with an amine in the presence of a coupling agent in a single screw reactor at a temperature in the range of 20-35° C. for a residence time in the range of 10 seconds to 300 seconds to obtain the peptides and amides.

In an embodiment of the present disclosure, the acid is selected from aliphatic acid and/or aromatic acid. The acid is a mono acid, a di-acid or a poly acid.

In an embodiment of the present disclosure, the amine is selected from primary amine and/or secondary amine, which may be further substituted.

In an embodiment of the present disclosure, the coupling agent is selected from a group consisting of 1-(3-Dimethylaminopropyl)-3-ethylcarbodiimide hydrochloride (EDC.HCl), N, N′-dicyclohexyl carbodiimide (DCC), 1-hydroxybenzotriazole (HOBt), 1,1′-carbonyldiimidazole (CDI), N, N′-diisopropylcarbodiimide (DIC) and combination thereof.

In an embodiment of the present disclosure, the single screw reactor rotates at a speed in the range of 20-250 rpm.

The screw reactors as described herein are conventionally used in the polymer industry for synthesis of polymers by melt polymerization and for extrusions. Its application in other domains is uncommon and no such proposals exist in the prior art. Here inventors have envisaged a solvent free process for the synthesis of amides and/or peptides using a screw reactor (such screw reactor is covered in one of co-pending Indian patent application of the inventor with application Ser. No. 201911035946, which is referred herein in part and not in entirety). The jacketed single-screw reactor may be constructed from Teflon, glass or any metal selected from stainless steel SS316, copper, haste alloy and such like for continuous flow reactions involving solids/slurries. Jacket may be made from glass or any polymeric material or metal.

The reactants in powder form are fed using two screw conveyers that help maintain the desired feed rate of individual substrates. The feed is given to another vertically aligned screw with downward flow to avoid any liquid due to shear thinning or melting settling even in the meniscus form or an inclined screw with downward flow direction. It is necessary to mention that having horizontal arrangement of the screw for reaction retains some liquid unless extremely close clearance is kept between the threads and the chamber wall. However, such a situation can create friction in the presence of reaction mass, which can lead to unsafe operations.

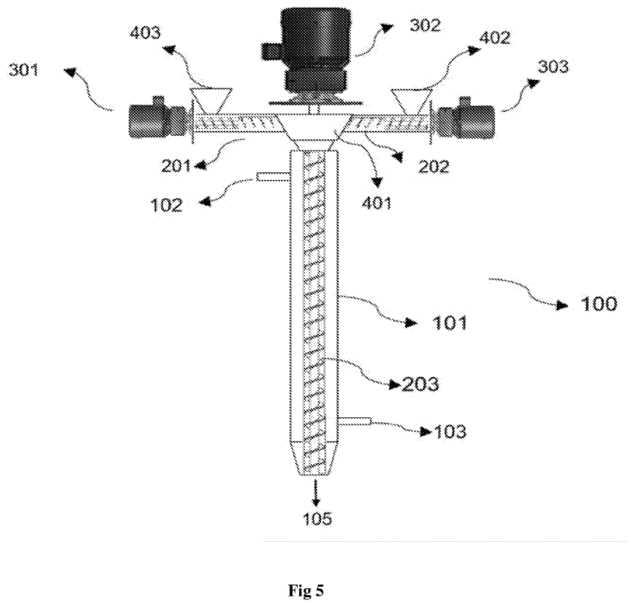

In the present disclosure, vertical alignment for screw reactor (100) (FIGS. 5A and 5B) (having glass or metal jacket) (101) such that the distance between the outer diameter of the screw thread and the inner diameter of the jacket is not more than 0.25 mm is preferred to avoid any liquid to remain accumulated. Any liquid used or generated in the reaction will flow downward due to gravity and any gas generated in the reaction will either flow in the form of packets or escape from the top if the material is not very densely packed. The inlet and outlet ports of the jacket (102 and 103), which may be alternated or swapped, are connected to a constant temperature circulation bath or wrapped with an electric 140 heating tape. For the reactions where reactants are in liquid or in slurry form, pumps were used for dosing instead of screw feeding system. This ensures no accumulation of reactants in the feed section to the reactor.

In another embodiment, the screw reactor of the present process comprises:
a) Jacketed screw reactor (100),
b) Jacket (101),
c) Outlet (102) for the heating/cooling fluid from jacket (101),
d) Inlet (103) for the heating/cooling fluid from jacket (101),
e) Feed funnel (401, 402, 403) connected to the screw reactor and to the two reactant dosing sections,
f) Outlet (105) of the screw reactor (100),
g) Reactant feeding screws (201, 202),
h) Screw for the reactor (203), and
i) Motors for screw rotation (301, 302, 303).

In specific embodiment, the screw reactor comprises combination of sequence of half reverse and complete forward threads, which is not covered in co-pending IN patent application Ser. No. 201911035946, and such arrangement of threads help in speeding up the reaction and making the residence time lesser to up to few seconds i.e., as low as 10 seconds, which ultimately reduces time for completion of reaction and products formation, and increasing overall throughput.

In an embodiment of the present disclosure, the process has greater than 95% conversion of the solid/liquid substrates and 45-95% and 49-95% yield of the desired peptides and amides respectively. The process produces at least 90% pure peptides and amides.

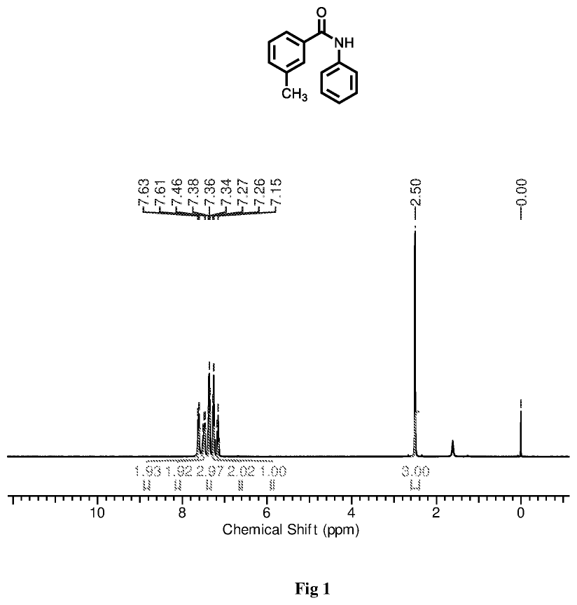

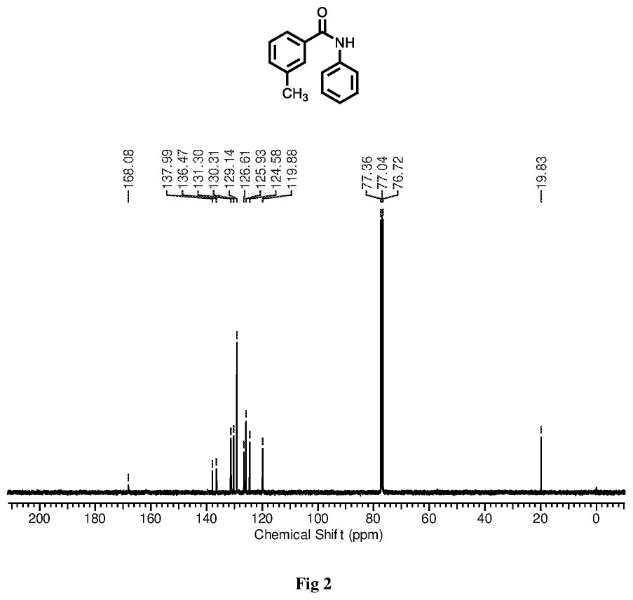

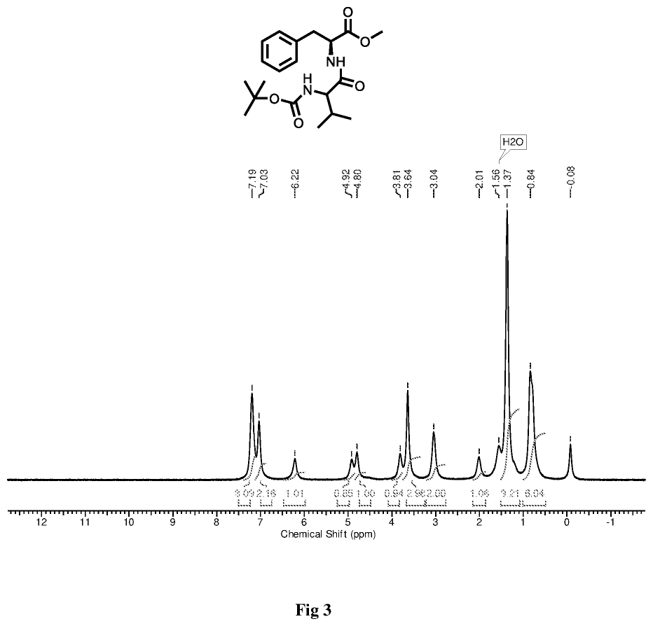

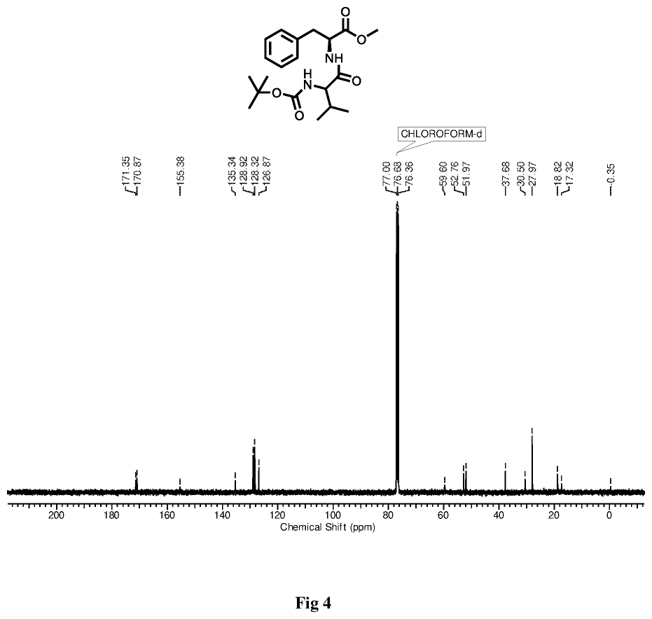

In a preferred embodiment, the solvent free continuous synthesis of amides and peptides is 90% more energy efficient than conventional batch operation. The purity of amides and peptides after work up is greater than 90% without needing any further purification, refer FIGS. 1-4.

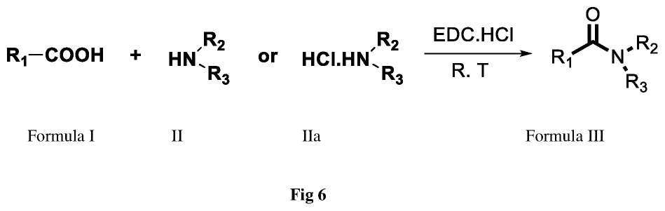

Accordingly, referring to FIG. 6, the acid of formula I and amine of formula II or IIa is reacted at room temperature to obtain the amide or peptide of formula III.

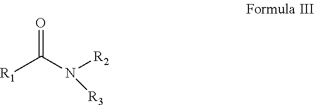

wherein,
R₁ is selected from aryl, alkyl, heteroaryl, substituted benzyl, unsaturated acids, further substituted acids and protected amino acids;
R₂ and R₃ may be same or different and selected from H, alkoxy, alkyl, aryl, heteroaryl, cyclic, acyclic, primary and secondary amine, with the proviso that only one of R₂ or R₃ may be H.

Table 1 enumerates the substrate scope of the room temperature process of the invention disclosing the various amides and peptides synthesized in the screw reactor.

TABLE 1
Amides and peptides (substrate scope).

| Sr. | Name of the starting | | Com- |
|---|---|---|---|
| No. | materials | Products | pounds |
| 1 | Benzoic acid + aniline | 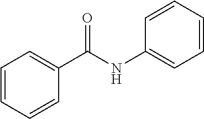 [SMILES: O=C(Nc1ccccc1)c1ccccc1] | 1a |
| 2 | 2,4- dimethoxybenzoic acid + aniline | 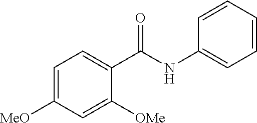 [SMILES: COc1ccc(C(=O)Nc2ccccc2)c(OC)c1] | 1b |
| 3 | 1-naphthoic acid + aniline | 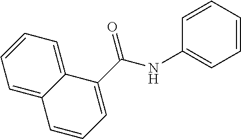 [SMILES: O=C(Nc1ccccc1)c1cccc2ccccc12] | 1c |
| 4 | Thiophene-2- carboxylic acid + aniline | 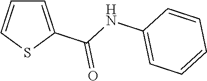 [SMILES: O=C(Nc1ccccc1)c1cccs1] | 1d |
| 5 | 2-phenylacetic acid + aniline | 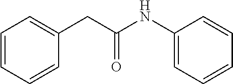 [SMILES: O=C(Cc1ccccc1)Nc1ccccc1] | 1e |
| 6 | 2-(4- isobutylphenyl) propanoic acid + aniline | 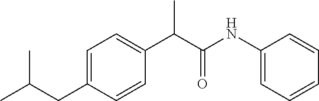 [SMILES: CC(C)Cc1ccc(C(C)C(=O)Nc2ccccc2)cc1] | 1f |
| 7 | 2-acetoxybenzoic acid + Aniline | 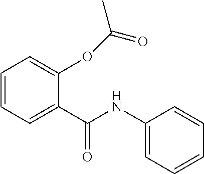 [SMILES: CC(=O)Oc1ccccc1C(=O)Nc1ccccc1] | 1g |
| 8 | (S)-2-(6- methoxynaphthalen- 2-yl)propanoic acid + aniline | 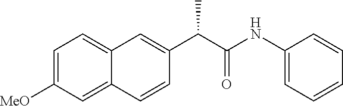 [SMILES: COc1ccc2cc([C@H](C)C(=O)Nc3ccccc3)ccc2c1] | 1h |
| 9 | Isonicotinic acid + aniline | 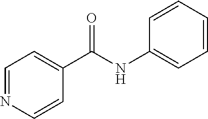 [SMILES: O=C(Nc1ccccc1)c1ccncc1] | 1i |
| 10 | Benzoic acid + diisobutylamine | 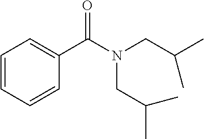 [SMILES: CC(C)CN(CC(C)C)C(=O)c1ccccc1] | 1j |
| 11 | Benzoic acid + dibenzylamine | 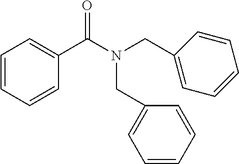 [SMILES: O=C(c1ccccc1)N(Cc1ccccc1)Cc1ccccc1] | 1k |
| 12 | Benzoic acid + morpholine | 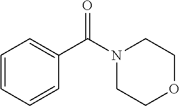 [SMILES: O=C(c1ccccc1)N1CCOCC1] | 1l |
| 13 | (E)-3-(4- methoxyphenyl) acrylic acid + morpholine | 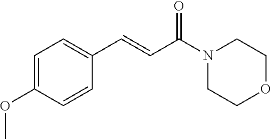 [SMILES: COc1ccc(/C=C/C(=O)N2CCOCC2)cc1] | 1m |
| 14 | Octanedioic acid + aniline | 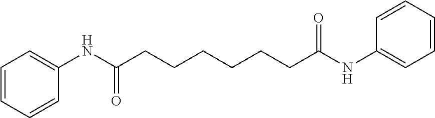 [SMILES: O=C(CCCCCCC(=O)Nc1ccccc1)Nc1ccccc1] | 1n |
| 15 | Octanedioic acid + o- toluidine | 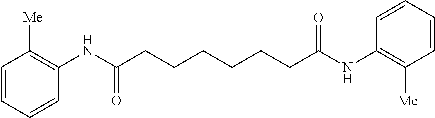 [SMILES: Cc1ccccc1NC(=O)CCCCCCC(=O)Nc1ccccc1C] | 1o |
| 16 | Octanedioic acid + 4- methoxyaniline | 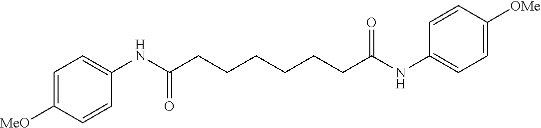 [SMILES: COc1ccc(NC(=O)CCCCCCC(=O)Nc2ccc(OC)cc2)cc1] | 1p |
| 17 | Nonanedioic acid + 3-nitroaniline | 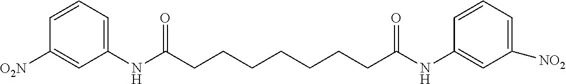 [SMILES: O=C(CCCCCCCC(=O)Nc1cccc([N+](=O)[O-])c1)Nc1cccc([N+](=O)[O-])c1] | 1q |
| 18 | Octanedioic acid + 4- chloro aniline | 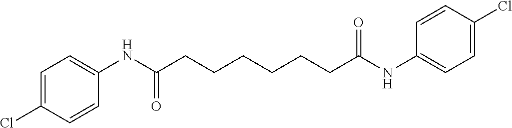 [SMILES: O=C(CCCCCCC(=O)Nc1ccc(Cl)cc1)Nc1ccc(Cl)cc1] | 1r |
| 19 | (E)-but-2-enoic acid + aniline | 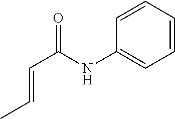 | 1s |
| 20 | (E)-but-2-enoic acid + morpholine | 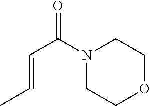 | 1t |
| 21 | Oleic acid + aniline | 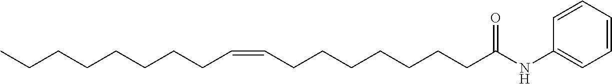 [SMILES: CCCCCCCC/C=C\CCCCCCCC(=O)Nc1ccccc1] | 1u |
| 22 | Nonanoic acid + aniline | 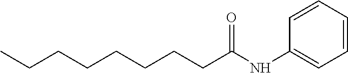 [SMILES: CCCCCCCCC(=O)Nc1ccccc1] | 1v |
| 23 | L-Phenylalanine methyl ester hydrochloride + Boc—Val—OH | 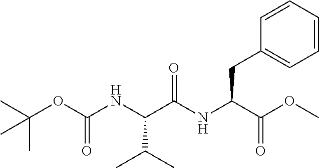 [SMILES: COC(=O)[C@H](Cc1ccccc1)NC(=O)[C@@H](NC(=O)OC(C)(C)C)C(C)C] | 1w |
| 24 | 3-methyl benzoic acid + aniline | 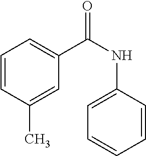 [SMILES: Cc1cccc(C(=O)Nc2ccccc2)c1] | 1y |

Thus the present disclosure provides a process for solvent free continuous synthesis of amides and peptides. The process avoids the use of solvent and converts the substrates in less residence time, which in turn makes the process economically suitable/sustainable. The process also involves stoichiometric quantities of reagents. The process of present disclosure includes other economic advantages, in terms of cost, energy efficient, environmentally friendliness as well as sustainability.

Differences wrt prior arts are: room temperature (25-30° C.) process (prior arts are at higher 60-70 deg C.), solvent less, continuous is novelty, very less time and no work-up. Extremely pure compounds are obtained. This is a versatile platform for solid-solid-, solid-liquid and liquid-liquid reactions, ideally solid-liquid works best. The present disclosure discloses a continuous process for synthesis of peptides and amides comprising reacting acids with amines at room temperature for 1-3 minutes to obtain greater than 90% pure products.

In an embodiment of the present invention, the process can be used for the synthesis of various drugs such as Ibuprofen, Naproxen, paracetamol and other drugs.

While the foregoing describes various embodiments of the disclosure, other and further embodiments of the disclosure may be devised without departing from the basic scope thereof. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

### General Examples for Solvent Free Continuous Synthesis of Amides and Peptides

Following examples are given by way of illustration and therefore should not be construed to limit the scope of the invention.

### Comparative Examples

### Example 1: Batch Examples With Solvent:

The benzoic acid (1 eq.) and aniline (1 eq.) with EDC.HCl (1.1 eq.) at 70° C. and acetonitrile solvent with 12 hours reaction time, reaction mass collected having greater than 99% conversion of substrate with average yield 62% and needs purification of column chromatography.

### Example 2: Batch Examples Without Solvent:

The benzoic acid (1 eq.) and aniline (1 eq.) with EDC.HCl (1.1 eq.) at 70° C. without using solvent with 12 hours reaction time, reaction mass collected having greater than 99% conversion of substrate with average yield 59% and needs purification by column chromatography.

### General Example 3:

The solid acids (1 eq.) and solid/liquid amines (1 eq.) with EDC.HCI (1.1 eq.) were continuously fed in to the vertical single screw reactor (Specification: SS316, I.D=1.27cm, length=30 cm, screw I.D=1.0 cm, length=29 cm), at room temperature (30° C.) with residence time of 10 s-300 sec.

The process resulted in the generation of amides and peptides at atmospheric temperature, and the reaction mass white in color (depends on substrate).

Upon mixing acids (1.0 eq.) and amines (1.0 eq.) with EDC.HCl (1.1 eq.) or DCC<CDI or HOBt in the absence of solvent in to the screw rector operated at room temperature with the screw rotation speed fixed at 20-250 rpm that resulted in a residence time of 300 s, the collected reaction mass was precipitated by water wash. Then filter the crude reaction mass on filter paper, dry in air and analyzed by NMR data, indicating of complete conversion of acids and amines. After completion of reaction and precipitation, the final amides and peptides was obtained in good to moderate yield. Then above reaction conditions were employed to the synthesis of amides and peptides that could be isolated in the 49% to 95% yield.

### NMR Data of Amides and Peptides (Compounds 1a-1w)

### Compound 1a: N-phenylbenzamide

Yield 95%, ¹H NMR (500 MHz, CHLOROFORM-d) □=7.99 (br. s, 1H), 7.89 (d, J=7.2 Hz, 2H), 7.68 (d, J=8.0 Hz, 2H), 7.57 (t, J=7.1 Hz, 1H), 7.49 (t, J=7.4 Hz, 2H), 7.39 (t, J=7.6 Hz, 2H), 7.18 (t, J=7.2 Hz, 1H).

¹³C NMR (126 MHz, CHLOROFORM-d) □=165.9, 138.0, 135.0, 131.8, 129.1, 128.8, 127.1, 124.6, 120.3.

Compound 1b: 2, 4-dimethoxy-N-phenylbenzamide

Yield 89%, ¹H NMR (500 MHz, CHLOROFORM-d) □=9.72 (br. s, 1H), 8.28 (d, J=8.8 Hz, 1H), 7.69 (d, J=8.0 Hz, 2H), 7.37 (t, J=7.8 Hz, 2H), 7.13 (t, J=7.4 Hz, 1H), 6.67 (dd, J=1.9, 8.8 Hz, 1H), 6.60-6.46 (m, 1H), 4.04 (s, 3H), 3.88 (s, 3H).

¹³C NMR (126 MHz, CHLOROFORM-d) □=163.8, 163.1, 158.6, 138.6, 134.2, 128.9, 123.9, 120.4, 114.7, 105.7, 98.8, 56.2, 55.6.

Compound 1c: N-phenyl-1-naphthamide

Yield 78%, ¹H NMR (500 MHz, CHLOROFORM-d) □=8.38 (d, J=7.2 Hz, 1H), 7.98 (d, J=8.0 Hz, 1H), 7.95-7.87 (m, 2H), 7.81 (br. s, 1H), 7.78-7.66 (m, 3H), 7.66-7.55 (m, 2 H), 7.51 (t, J=7.6 Hz, 1H), 7.42 (t, J=7.2 Hz, 2H), 7.21 (t, J=7.2 Hz, 1H).

¹³C NMR (126 MHz, CHLOROFORM-d) □=167.6, 138.1, 134.5, 133.8, 131.1, 130.1, 129.2, 128.4, 127.4, 126.6, 125.3, 125.1, 124.8, 124.7, 120.0.

Compound 1d: N-phenylthiophene-2-carboxamide

Yield 92%, ¹H NMR (500 MHz, CHLOROFORM-d) □=7.93 (br. s, 1H), 7.79-7.60 (m, 3H), 7.56 (d, J=4.2 Hz, 1H), 7.37 (t, J=7.2 Hz, 2H), 7.24-7.00 (m, 2H).

¹³C NMR (126 MHz, CHLOROFORM-d) □=160.1, 139.3, 137.6, 130.8, 129.1, 128.5, 127.8, 124.6, 120.3.

Compound 1e: N, 2-diphenylacetamide

Yield 89%, ¹H NMR (500 MHz, CHLOROFORM-d) □=7.46 (d, J=8.0 Hz, 3H), 7.43-7.39 (m, 2H), 7.36 (d, J=6.1 Hz, 3H), 7.30 (t, J=7.4 Hz, 2H), 7.20-7.03 (m, 1H), 3.74 (s, 2H).

¹³C NMR (126 MHz, CHLOROFORM-d) □=169.3, 137.7, 134.5, 129.5, 129.2, 128.9, 127.6, 124.5, 119.9, 44.8.

Compound 1f: 2-(4-isobutylphenyl)-N-phenylpropanamide

Yield 82%, ¹H NMR (500 MHz, CHLOROFORM-d) □=7.44 (d, J=7.6 Hz, 2H), 7.29 (br. s, 4H), 7.18 (d, J=7.2 Hz, 2H), 7.14-6.92 (m, 2H), 3.72 (d, J=6.9 Hz, 1H), 2.50 (d, J=6.9 Hz, 2H), 2.00-1.79 (m, 1H), 1.62 (d, J=6.9 Hz, 3H), 0.94 (d, J=6.5 Hz, 6H).

¹³C NMR (126 MHz, CHLOROFORM-d) □=172.6, 141.1, 138.0, 137.9, 129.9, 128.9, 127.5, 124.2, 119.6, 47.8, 45.0, 30.2, 22.4, 18.5.

Compound 1g: 2-(phenylcarbamoyl) phenyl acetate

Yield 89%, ¹H NMR (500 MHz, CHLOROFORM-d) □=8.06 (br. s, 1H), 7.88 (d, J=7.6 Hz, 1H), 7.63 (d, J=8.0 Hz, 2H), 7.54 (d, J=7.6 Hz, 1H), 7.39 (t, J=6.9 Hz, 3H), 7.23-7.12 (m, 2H), 2.36 (s, 3H).

¹³C NMR (126 MHz, CHLOROFORM-d) □=169.3, 163.6, 147.7, 137.8, 132.2, 130.0, 129.2, 126.6, 124.7, 123.4, 121.2, 119.9, 21.1.

Compound 1h: (S)-2-(6-methoxynaphthalen-2-yl)-N-phenylpropanamide

Yield 69%, ¹H NMR (500 MHz, CHLOROFORM-d) □=7.84-7.70 (m, 3H), 7.54-7.36 (m, 3H), 7.32-7.25 (m, 2H), 7.25-7.13 (m, 3H), 7.12-7.00 (m, 1H), 4.00-3.92 (m, 3H), 3.88 (d, J=6.9 Hz, 1H), 1.70 (d, J=7.2 Hz, 3H).

¹³C NMR (126 MHz, CHLOROFORM-d) □=172.5, 157.9, 137.9, 136.0, 133.9, 129.3, 129.0, 128.9, 127.9, 126.3, 126.2, 124.2, 119.7, 119.3, 105.7, 55.4, 48.1, 18.6.

### Compound 1i: N-phenylisonicotinamide

Yield 71%, ¹H NMR (500 MHz, CHLOROFORM-d) □=8.77 (d, J=4.6 Hz, 2H), 8.10 (br. s, 1H), 7.71 (d, J=5.3 Hz, 2H), 7.65 (d, J=7.6 Hz, 2H), 7.39 (t, J=7.8 Hz, 2H), 7.25-7.10 (m, 1H).

¹³C NMR (126 MHz, CHLOROFORM-d) □=163.8, 150.7, 142.1, 137.2, 129.2, 125.3, 120.9, 120.4.

### Compound 1j: N, N-diisobutylbenzamide

Yield 79%, ¹H NMR (500 MHz, CHLOROFORM-d) □=7.41-7.29 (m, 5 H), 3.34 (d, J=6.9 Hz, 2H), 3.08 (d, J=6.1 Hz, 2H), 2.16-2.04 (m, 1H), 1.82 (br. s, 1H), 0.97 (d, J=5.3 Hz, 6H), 0.81-0.58 (m, 6H).

¹³C NMR (126 MHZ, CHLOROFORM-d) □=172.5, 137.5, 129.0, 128.4, 128.3, 127.0, 126.4, 56.6, 51.2, 44.6, 26.8, 26.2, 20.2, 19.8.

### Compound 1k: N, N-dibenzylbenzamide

Yield 86%, ¹H NMR (500 MHZ, CHLOROFORM-d) □=7.52 (br. s, 2 H), 7.47-7.30 (m, 11H), 7.17 (br. s, 2H), 4.73 (br. s, 2H), 4.42 (br. s, 2H).

¹³C NMR (126 MHz, CHLOROFORM-d) □=172.2, 136.9, 136.4, 136.1, 129.6, 128.8, 128.7, 128.5, 128.4, 127.6, 127.0, 126.7, 51.5, 46.8.

Compound 1l: morpholino (phenyl) methanone

Yield 82%, ¹H NMR (500 MHz, CHLOROFORM-d) □=7.34 (br. s, 5 H), 3.75-3.52 (m, 6H), 3.38 (br. s, 2H).

¹³C NMR (126 MHZ, CHLOROFORM-d) □=170.2, 135.0, 129.6, 128.3, 126.8, 66.6.

Compound 1m: (E)-3-(4-methoxyphenyl)-1-morpholinoprop-2-en-1-one

Yield 54%, ¹H NMR (500 MHz, CHLOROFORM-d) □=7.66 (d, J=15.3 Hz, 1H), 7.55-7.37 (m, J=8.0 Hz, 2H), 7.00-6.81 (m, J=8.4 Hz, 2H), 6.71 (d, J=15.3 Hz, 1H), 3.83 (s, 3H), 3.78 (br. s, 1H), 3.71 (br. s, 7H).

¹³C NMR (126 MHz, CHLOROFORM-d) □=165.9, 161.0, 142.9, 129.4, 127.9, 114.2, 114.0, 66.9, 55.4.

Compound 1n: N1, N8-diphenyloctanediamide

Yield 87%, ¹H NMR (400 MHZ, DMSO-d6) □=9.85 (br. s, 2H), 7.59 (d, J=7.6 Hz, 4H), 7.27 (t, J=7.6 Hz, 4H), 7.13-6.85 (m, 2H), 2.30 (t, J=7.1 Hz, H), 1.60 (br. s, 4H), 1.33 (br. s, 4H).

¹³C NMR (101 MHz, DMSO-d6) □=171.6, 139.8, 129.0, 123.3, 119.4, 36.8, 28.9, 25.4.

Compound 1o: N1, N8-di-o-tolyloctanediamide

Yield 82%, ¹H NMR (400 MHZ, DMSO-d6) □=9.23 (br. s, 2H), 7.35 (d, J=7.6 Hz, 2H), 7.19 (d, J=7.3 Hz, 2H), 7.14 (t, J=7.3 Hz, 2H), 7.10-7.00 (m, 2H), 2.33 (t, J=7.3 Hz, 4H), 2.18 (s, 6H), 1.62 (br. s, 4H), 1.37 (br. s, 4H).

¹³C NMR (101 MHz, DMSO-d6) □=171.5, 136.9, 132.2, 130.6, 126.2, 125.6, 125.4, 36.2, 28.9, 25.7, 18.3.

Compound 1p: N1, N8-bis (4-methoxyphenyl) octanediamide

Yield 85%, ¹H NMR (400 MHZ, DMSO-d6) □=9.70 (s, 2H), 7.48 (d, J=9.0 Hz, 4H), 6.85 (d, J=9.0 Hz, 4H), 3.70 (s, 6H), 2.25 (t, J=7.4 Hz, 4H), 1.70-1.47 (m, 4H), 1.32 (br. s, 4H).

¹³C NMR (101 MHz, DMSO-d₆) □=170.9, 155.2, 132.7, 120.8, 113.9, 55.3, 36.4, 28.7, 25.3.

Compound 1q: N1, N9-bis (3-nitrophenyl) nonanediamide

Yield 57%, ¹H NMR (400 MHz, DMSO-d6) □=9.70 (s, 2H), 7.49 (d, J=8.9 Hz, 4H), 6.85 (d, J=8.9 Hz, 4H), 3.70 (s, 6H), 2.25 (t, J=7.3 Hz, 4H), 1.57 (br. s, 4H), 1.30 (br. s, 7H).

¹³C NMR (101 MHz, DMSO-d6) □=170.7, 155.0, 132.5, 120.5, 113.7, 55.1, 36.3, 28.7, 28.6, 25.2.

Compound 1r: N1, N8-bis (4-chlorophenyl) octanediamide

Yield 78%, ¹H NMR (400 MHZ, DMSO-d₆) □=9.98 (s, 2H), 7.61 (d, J=8.6 Hz, 4H), 7.32 (d, J=8.5 Hz, 4H), 2.29 (t, J=7.3 Hz, 4H), 1.59 (br. s, 4H), 1.44-1.14 (m, 6H).

¹³C NMR (101 MHz, DMSO-d6) □=171.6, 138.5, 128.7, 126.6, 120.7, 36.5, 28.7, 25.1.

Compound 1s: (E)-N-phenylbut-2-enamide

Yield 69%, ¹H NMR (500 MHz, CHLOROFORM-d) □=7.65 (br. s, 1 H), 7.57 (d, J=8.0 Hz, 2H), 7.33 (t, J=7.8 Hz, 2H), 7.12 (t, J=7.4 Hz, 1H), 5.79 (s, 1H), 5.46 (s, 1H), 2.06 (s, 3H).

¹³C NMR (126 MHz, CHLOROFORM-d) □=166.4, 140.6, 137.5, 128.7, 124.1, 119.8, 119.5, 18.5.

Compound 1t: (E)-1-morpholinobut-2-en-1-one

Yield 56%, ¹H NMR (500 MHz, CHLOROFORM-d) □=5.00 (br. s, 1H), 4.81 (s, 1H), 3.44 (br. s, 4H), 3.38 (br. s, 4H), 1.73 (s, 3H).

¹³C NMR (126 MHz, CHLOROFORM-d) □=170.8, 139.6, 115.4, 66.5, 20.1.

### Compound 1u: N-phenyloleamide

Yield 92%, ¹H NMR (500 MHz, CHLOROFORM-d) □=7.76 (br. s, 1H), 7.56 (d, J=7.6 Hz, 2H), 7.31 (t, J=7.6 Hz, 2H), 7.21-7.03 (m, 1H), 5.36 (d, J=2.3 Hz, 2H), 2.69 (s, 1H), 2.37 (t, J=7.4 Hz, 2H), 2.03 (d, J=5.7 Hz, 4H), 1.80-1.59 (m, 2H), 1.29 (s, 11H), 1.32 (s, 8H), 0.90 (t, J=6.7 Hz, 3H).

¹³C NMR (126 MHz, CHLOROFORM-d) □=172.0, 138.3, 130.2, 129.9, 129.1, 124.3, 120.1, 43.2, 37.9, 32.1, 30.0, 29.9, 29.7, 29.5, 29.3, 27.4, 25.9, 22.9, 14.3.

### Compound 1v: N-phenylnonanamide

Yield 88%, ¹H NMR (500 MHz, CHLOROFORM-d) □=7.65-7.42 (m, 3H), 7.41-7.26 (m, 2H), 7.11 (t, J=7.4 Hz, 1H), 2.37 (t, J=7.6 Hz, 2H), 1.74 (quin, J=7.3 Hz, 2H), 1.47-1.34 (m, 2H), 1.30 (dd, J=5.1, 10.5 Hz, 8H), 0.90 (t, J=6.7 Hz, 3H).

¹³C NMR (126 MHz, CHLOROFORM-d) □=171.9, 138.2, 129.2, 124.3, 120.1, 38.0, 32.0, 29.6, 29.5, 29.4, 25.9, 22.9, 14.3.

Compound 1w: methyl (tert-butoxycarbonyl)-L-valyl-L-phenylalaninate

Yield 49%, ¹H NMR (400 MHZ, CHLOROFORM-d) □=7.19 (br. s, 3H), 7.03 (br. s, 2H), 6.22 (br. s, 1H), 4.92 (br. s, 1H), 4.80 (br. s, 1H), 3.81 (br. s, 1H), 3.64 (br. s, 3H), 3.04 (br. s, 2H), 2.01 (br. s, 1H), 1.37 (br. s, 9H), 0.84 (br. s, 6H).

¹³C NMR (101 MHz, CHLOROFORM-d) □=171.3, 170.9, 155.4, 135.3, 128.9, 128.3, 126.9, 59.6, 52.8, 52.0, 37.7, 30.5, 28.0, 18.8, 17.3.

Compound 1y: 3-methyl-N-phenylbenzamide

Yield: (White solid, 86%)

¹H NMR (400 MHZ, CHLOROFORM-d) □=7.62 (d, J=7.6 Hz, 2H), 7.55-7.43 (m, 2H), 7.41-7.31 (m, 3H), 7.30-7.24 (m, 2H), 7.18-7.12 (m, 1H), 2.50 (s, 3H).

¹³C NMR (101 MHz, CHLOROFORM-d) □=168.1, 138.0, 136.5, 131.3, 130.3, 129.1, 126.6, 125.9, 124.6, 119.9, 19.8.

### Example 4: Screw Reactor Used in the Process:

A jacketed single-screw reactor (jacket-screw materials: Glass-Teflon or glass-metal or metal-metal) have used for the continuous flow mechanochemical synthesis of amides and/or peptides. Typical screw can be kept vertical or horizontal or inclined (outlet positioned at height below the inlet level) alignment for the screw reactor having a glass jacket with 42 mm outer diameter and straight annulus diameter of 18 mm, which is also the inner diameter that houses the screw. PTFE Screw length is 340 mm with 17.5 mm diameter. This leaves a gap of only 0.25 mm between the jacket wall and the screw threads, the screw reactor as shown in FIG. 5. The inlet and outlet ports of the jacket are connected to a constant temperature circulation bath. The residence time was controlled using the rotation speed of the screw, controlled using a precision motor. The screw reactor parameters can be tuned to optimize the process for the amide synthesis with good to excellent yield with short residence time including the screw profile, feed rate, screw speed, and temperature. Furthermore, various output parameters can be monitored both during and after the reactive extrusion, and these parameters includes throughput rate, which is the amount of product produced (per hour or per day) and residence time, which is the time required for the solid material to pass through the using screw extruder (from a few second to few minutes) depending on screw speed. For a few experiments to check the reproducibility, experiments were carried out using tiny screw reactors (diameter 10 mm, screw pitch 2 mm, screw depth 2 mm and screw height 200 mm). For scale-up experiments a 20 ml volume screw reactor (diameter 25.4 mm, screw pitch 10 mm, screw depth 2 mm and screw height 400 mm) was used.

The foregoing descriptions of specific embodiments of the present invention have been presented for purposes of illustration and description. They are not intended to be exhaustive or to limit the present invention to the precise forms disclosed, and obviously, many modifications and variations are possible in the light of the above teaching.

The embodiments were chosen and described to best explain the principles of the present invention and its practical application, to thereby enable others skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

### ADVANTAGES OF THE INVENTION

Solvent free continuous synthesis
Zero discharge process
Process provides good yield and high purity of products
Time for reaction completion is substantially reduced
Purification free process

1. A continuous process for synthesizing peptides and/or amides, the continuous process comprising:
reacting an acid with an amine in the presence of a coupling agent in a single screw reactor at a temperature from 20° C. to 35° C. for a residence time from 10 seconds to 300 seconds to obtain the peptides and/or amides.

2. The continuous process according to claim 1. wherein the acid is selected from aliphatic acid and/or aromatic acid.

3. The continuous process according to claim 2. wherein the acid is a mono acid, a di-acid, or a poly acid.

4. The continuous process according to claim 1. wherein the amine is selected from primary amine and/or secondary amine.

5. The continuous process according to claim 1. wherein the coupling agent is selected from a group consisting of 1-(3-Dimethylaminopropyl)-3-ethylcarbodiimide hydrochloride, N, N′-dicyclohexyl carbodiimide (DCC), 1-hydroxybenzotriazole, 1,1′-carbonyldiimidazole, N, N′-diisopropylcarbodiimide, and combinations thereof.

6. The continuous process according to claim 1, wherein the single screw reactor rotates at a speed from 20 rpm to 250 rpm.

7. The continuous process according to claim 1, wherein a conversion rate of the solid/liquid substrates into the amides and/or peptides is from 95% to 100%.

8. The continuous process according to claim 1. wherein yield of the peptides and/or amides is from 45% to 95%.

9. The continuous process according to claim 1, wherein purity of the amides and/or peptides is from 95% to 99%.

10. The continuous process according to claim 1, wherein the continuous process is a solvent free continuous process.

11. The continuous process according to claim 1, wherein the continuous process is a purification free continuous process.
